(19) United States
(12) Patent Application Publication
(10) Pub. No.: US 2022/0283149 A1
(43) Pub. Date: Sep. 8, 2022

(54) ALL-IN-ONE MOLECULAR DIAGNOSTICS DEVICE

(71) Applicant: GMD BIOTECH, INC, Gwangju (KR)

(72) Inventors: Min Gon KIM, Gwangju (KR); Young Ung SEOK, Gwangju (KR)

(73) Assignee: GMD BIOTECH, INC, Gwangju (KR)

(21) Appl. No.: 17/636,004

(22) PCT Filed: Aug. 5, 2020
(86) PCT No.: PCT/KR2020/010321

§ 371 Date: Feb. 16, 2022

(30) Foreign Application Priority Data
Aug. 6, 2019 (KR) 10-2019-0095588

(51) Int. Cl. G01N 33/52; C12Q 1/6851

(52) U.S. Cl. CPC G01N 33/525; C12Q 1/6851

(57) ABSTRACT

Disclosed is an all-in-one molecular diagnostics device, and more particularly, a molecular diagnostics device comprising a binding pad specifically binding to a diagnostic target molecule, a transfer pad including an elution buffer in a dry state that separates the diagnostic target molecule from the binding pad, and an amplification pad where an amplification reaction for the diagnostic target molecule occurs.

### BACKGROUND OF THE INVENTION

### Field of the Invention

The present disclosure relates to an all-in-one molecular diagnostics device, and more particularly, to a molecular diagnostics device comprising a binding pad specifically binding to a diagnostic target molecule, a transfer pad including an elution buffer in a dry state that separates the diagnostic target molecule from the binding pad, and an amplification pad where an amplification reaction for the diagnostic target molecule occurs.

### Description of the Related Art

Molecular diagnosis or molecular diagnostics refers to a diagnostic field or technique that detects or analyzes biomarkers (particularly, DNA or RNA) using molecular biological technology and particularly, has been used as a meaning similar to nucleic acid diagnosis.

Currently, a method mainly used for virus detection is a molecular diagnostics method that detects a cause of disease and infection by detecting nucleic acids (DNA and RNA or variants thereof) of bacteria, viruses, and the like that cause diseases. The molecular diagnostics method consists of four steps: collecting a sample from a body fluid, extracting a gene from the collected sample, and amplifying and analyzing the gene using a polymerase chain reaction. Since the molecular diagnostics method is subjected to a gene amplification process, accurate diagnosis with very high sensitivity and specificity is possible even for trace pathogens.

However, since expensive analysis equipment and reagents such as PCR and electrophoresis are used to perform conventional diagnostics methods, cost is expensive and complex and specialized techniques are required, so that the conventional diagnostics methods can be performed only by skilled technicians. In addition, since it is difficult to integrate each biological reaction step due to the hugeness of the analysis equipment, there is always a possibility of sample contamination in the molecular diagnosis process, and since the analysis time is long, there is a limit to genetic diagnosis in the field.

The above-described technical configuration is the background art for helping in the understanding of the present invention, and does not mean a conventional technology widely known in the art to which the present invention pertains.

### SUMMARY OF THE INVENTION

The present disclosure is invented to solve the aforementioned problems, and an object of the present disclosure is to provide a molecular diagnostics device comprising a binding pad specifically binding to a diagnostic target molecule, a transfer pad including an elution buffer in a dry state that separates the diagnostic target molecule from the binding pad, and an amplification pad where an amplification reaction for the diagnostic target molecule occurs.

Further, an object of the present disclosure is to provide a molecular diagnostics device for generating an amplification reaction by capturing a diagnostic target molecule carried by a washing buffer using a binding pad itself or a binding material included in the binding pad in a dry state, and separating the diagnostic target molecule captured on the binding pad or the binding material of the binding pad from the binding material using an elution buffer included in a transfer pad in a dry state.

Objects of the present disclosure are not limited to the objects described above, and other objects, which are not mentioned above, will be apparent from the following description.

An aspect of the present disclosure provides a molecular diagnostics device comprising an application pad, wherein the amplification pad may include a binding pad specifically binding to a diagnostic target molecule, a transfer pad including an elution buffer in a dry state that separates the diagnostic target molecule from the binding pad.

The binding pad may include a porous membrane and the porous membrane may be at least one selected from the group consisting of glass fiber, silica membrane, cellulose, nitrocellulose, cellulose acetate, cotton, and nylon, but is not limited thereto.

The binding pad may further include a binding material that specifically binds to the diagnostic target molecule, and the binding material may be included in the binding pad in a dry state.

The binding material may be at least one selected from the group consisting of nucleic acid, aptamer, hapten, locked nucleic acid (LNA), antibody, antigen, DNA or RNA binding protein, single strand binding protein, Rec A protein, polypeptide, Fab fragment small-molecular chemical material, cationic compound, synthetic polymer, and mixtures thereof, but is not limited thereto.

The cationic compound may be a cationic lipid or cationic polymer, but is not limited thereto.

The amplification pad may further include a channel pad and a reaction pad in addition to the binding pad and the transfer pad.

According to an embodiment of the present disclosure, the amplification pad may include a binding pad that includes a binding material specifically binding to a diagnostic target molecule and specifically binds to the diagnostic target molecule transferred by a washing buffer to extract the diagnostic target molecule; a transfer pad disposed on an upper surface of the binding pad and receiving the diagnostic target molecule separated from the binding pad; a channel pad disposed on an upper surface of the transfer pad and including at least one molecule passage channel through which the diagnostic target molecule received from the transfer pad passes; and at least one reaction pad disposed at a position corresponding to the at least one molecule passage channel and generating an amplification reaction for the diagnostic target molecule.

The all-in-one molecular diagnostics device of the present disclosure may further include a sample pad, a loading pad, and a wicking pad in addition to the amplification pad. The sample pad may accommodate a sample containing the diagnostic target molecule, the loading pad may accommodate a washing buffer carrying the diagnostic target molecule, and the wicking pad may be positioned downstream of the amplification pad and absorb the washing buffer developed into the amplification pad.

The loading pad, the sample pad, the amplification pad, and the wicking pad may be sequentially spaced apart from each other and arranged.

The washing buffer may include at least one of distilled water (D.W.) and deionized water (D.I water), but is not limited thereto.

The reaction pad may include a dry primer for generating the amplification reaction for the diagnostic target molecule, but is not limited thereto.

At least one of the transfer pad and the reaction pad may include a dry indicator of which fluorescence intensity changes as the amplification reaction occurs, but is not limited thereto.

At least one of the transfer pad and the reaction pad may include a dry amplification reaction enzyme for generating the amplification reaction for the diagnostic target molecule, but is not limited thereto.

The reaction pad may include dry (NH₄)₂SO₄ for forming a structure of the diagnostic target molecule, but is not limited thereto.

The transfer pad may include dry KCl for forming the structure of the diagnostic target molecule, but is not limited thereto.

The amplification pad may include a dry surfactant for accelerating the amplification reaction, but is not limited thereto.

The transfer pad may include dry betaine for accelerating the amplification reaction, but is not limited thereto.

The reaction pad may include dry MgSO₄ for controlling the fluorescence intensity as the amplification reaction occurs, but is not limited thereto.

At least one of the transfer pad and the reaction pad may include dry dNTPs for forming a synthesis block of the diagnostic target molecule, but is not limited thereto.

The transfer pad may be formed of a membrane having a structure in which the pore sizes decrease in a lower direction, but is not limited thereto.

Specific matters for achieving the above objects will be apparent with reference to embodiments to be described below in detail together with the accompanying drawings.

However, the present disclosure is not limited to embodiments to be disclosed below, but may be configured in various different forms, and will be provided to make the disclosure of the present disclosure complete and fully notify the scope of the present disclosure to persons with ordinary skill in the art to which the inventions pertain (hereinafter, “those skilled in the art”). According to the embodiment of the present disclosure, it is possible to perform separation of a diagnostic target molecule and high-sensitivity isothermal amplification reaction and detection, by capturing a diagnostic target molecule carried by a washing buffer using a binding pad itself or a binding material included in the binding pad in a dry state, separating the diagnostic target molecule captured on the binding material of the binding pad using an elution buffer included in a transfer pad in a dry state, moving the separated diagnostic target molecule to a reaction pad through a channel pad, and generating an amplification reaction of the diagnostic target molecule in the reaction pad.

The effects of the present disclosure are not limited to the above-described effects, and it will be understood that provisional effects toe expected by technical features of the present disclosure will be apparent from the following description.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a diagram illustrating a molecular diagnostics device according to an embodiment of the present disclosure;

FIGS. 2A and 2B are diagrams illustrating an amplification pad of the molecular diagnostics device according to an embodiment of the present disclosure;

FIG. 3 is a diagram illustrating an example of binding of chitosan and a diagnostic target molecule according to an embodiment of the present disclosure;

FIG. 4 is a diagram illustrating an example of an amplification reaction according to an embodiment of the present disclosure; and

FIG. 5 is a diagram illustrating molecular fluorescence images and fluorescence intensity graphs according to a configuration of a transfer pad according to an embodiment of the present disclosure.

### DETAILED DESCRIPTION OF THE PREFERRED EMBODIMENT

The present disclosure may have various modifications and various embodiments and specific embodiments will be illustrated in the drawings and described in detail.

Various features of the invention disclosed in the appended claims will be better understood in consideration of the drawings and the detailed description. Apparatuses, methods, manufacturing methods and various embodiments disclosed in the specification will be provided for illustrative purposes. The disclosed structural and functional features are intended to allow those skilled in the art to be specifically implemented in various embodiments, but are not intended to limit the scope of the invention. The disclosed terms and sentences are intended to be easily explained to the various features of the disclosed invention, but are not intended to limit the scope of the invention.

In describing the present disclosure, the detailed description of related known technologies will be omitted if it is determined that they unnecessarily make the gist of the present disclosure unclear.

Hereinafter, an all-in-one molecular diagnostics device according to an embodiment of the present disclosure will be described. Here, for example, a diagnostic target molecule may include a compound to be detected through an all-in-one molecular diagnostics device of the present disclosure, for example, at least one of a nucleic acid or a charged molecule, but is not limited thereto.

The nucleic acid is a genetic material of a living organism composed of monomers, which are nucleotides consisting of phosphate groups and bases, and typically includes deoxyribonucleic acid (DNA) and ribonucleic acid (RNA). More specifically, deoxyribonucleic acid including DNA includes DNA, mtDNA, cDNA, and the like, and ribonucleic acid including RNA includes RNA, mRNA, tRNA, rRNA, ncRNA, sgRNA, shRNA, siRNA, snRNA, miRNA, snoRNA, LNA, and the like, and also includes other nucleic acid analogs such as GNA, PNA, TNA, morpholino, and the like.

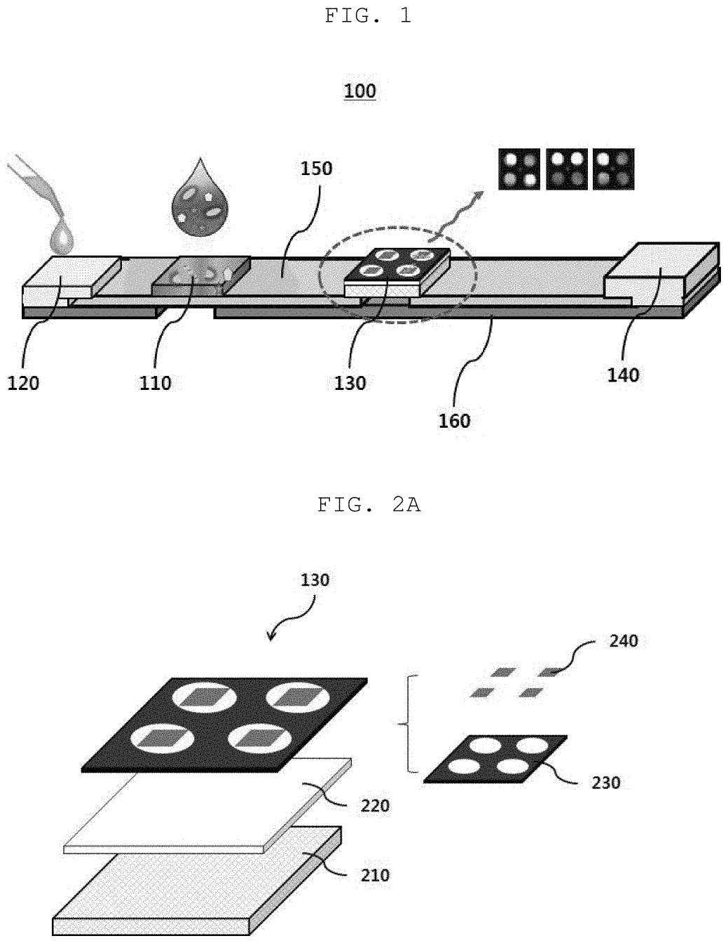

FIG. 1 is a diagram illustrating a molecular diagnostics device 100 according to an embodiment of the present disclosure.

Referring to FIG. 1, the molecular diagnostics device 100 includes a sample pad 110, a loading pad 120, an amplification pad 130, a wicking pad 140, a connection pad 150, and a support 160.

The sample pad 110 may accommodate a sample including a diagnostic target molecule. In an embodiment, the sample pad 110 includes a sample inlet, and the sample may be injected into the sample pad 110 through the sample inlet. Here, the sample may be a biological material, for example, a biological fluid or a biological tissue to extract the diagnostic target molecule. Examples of the biological fluid may include urine, blood (whole blood), plasma, serum, saliva, semen, stool, sputum, cerebrospinal fluid, tear, mucus, amniotic fluid, etc. The biological tissue is a cluster of cells, and may correspond to connective tissue, epithelial tissue, muscular tissue, neural tissue, etc., as a specific type of set with intracellular substances which typically form one of structural substances of human, animal, plant, bacteria, fungal or viral structures. In addition, examples of the biological tissue may also include organs, tumors, lymph nodes, arteries, and individual cell(s).

In an embodiment, the sample pad 110 may be treated to have alkaline. For example, the sample pad 110 may include a lysis buffer that destroys target cells in the sample to release the diagnostic target molecule from the target cells. Accordingly, when the sample is injected into the sample pad 110, the diagnostic target molecule to be detected may be leaked from the sample.

The loading pad 120 may accommodate a washing buffer carrying the diagnostic target molecule leaked from the sample accommodated in the sample pad 110. In an embodiment, the loading pad 120 includes a buffer inlet, and the washing buffer may be injected into the sample pad 110 through the buffer inlet. For example, the washing buffer may include at least one of distilled water (D.W.) and deionized water (D.I water).

The amplification pad 130 includes a binding pad 210 that specifically binds to the diagnostic target molecule or a binding material included in the binding pad 210 and an elution buffer in a dry state for separating the diagnostic target molecule from the binding material, and an application reaction for amplifying the separated diagnostic target molecule may occur through an amplification reagent. In an embodiment, the amplification pad 130 may extract or capture the diagnostic target molecule carried by the washing buffer using the binding pad 210 included in the amplification pad 130 or the binding material included in the binding pad 210. In addition, the amplification pad 130 separates the extracted or captured diagnostic target molecule using the elution buffer included in the amplification pad 130, and the amplification reaction may occur by the amplification reagent.

In a specific embodiment, when the diagnostic target molecule is a nucleic acid molecule, the amplification may mean the formation of a plurality of copies for the nucleic acid molecule or a plurality of copies complementary to the nucleic acid molecule using at least one of nucleic acid molecules as a template, and various known amplification techniques may be used.

The wicking pad 140 may absorb the washing buffer developed into the amplification pad 130. The wicking pad 140 may be positioned downstream of the amplifying pad 130. Accordingly, the washing buffer may be absorbed into the wicking pad 140 from the sample pad 110 through the amplification pad 130.

In an embodiment, each of the sample pad 110, the loading pad 120, the amplification pad 130, and the wicking pad 140 may be formed of a porous membrane. For example, a porous material constituting the porous membrane may include fibrous paper, a microporous membrane made of a cellulose material, cellulose, a cellulose derivative such as cellulose acetate, nitrocellulose, glass fiber, cotton, or a porous gel such as nylon, but is not limited thereto.

When each of the sample pad 110, the loading pad 120, the amplification pad 130, and the wicking pad 140 is formed of the porous membrane, the pads have liquid absorption to the washing buffer. In this case, the absorption of each of the sample pad 110, the loading pad 120, the amplification pad 130, and the wicking pad 140 may be different from each other. For example, the liquid absorption of the loading pad 120 may be the lowest, and the liquid absorption of the wicking pad 140 may be the largest. That is, the liquid absorption may be increased toward the sample pad 110, the loading pad 120, the amplification pad 130, and the wicking pad 140.

The molecular diagnostics device 100 according to the present disclosure may include a structure in which the loading pad 120, the sample pad 110, the amplification pad 130, and the wicking pad 140 are sequentially spaced apart from each other and arranged. That is, the molecular diagnostics device 100 according to the present disclosure may include a structure in which a plurality of pads are arranged in order of liquid absorption. Accordingly, due to this structure, the washing buffer injected into the loading pad 120 may be developed toward the wicking pad 140.

The connection pad 150 may connect the loading pad 120, the sample pad 110, the amplification pad 130, and the wicking pad 140 to move the washing buffer. In an embodiment, the connection pads 150 may be positioned between the loading pad 120 and the sample pad 110, between the sample pad 110 and the amplification pad 130, and between the amplification pad 130 and the wicking pad 140, respectively.

The support 160 may support the loading pad 120, the sample pad 110, the amplification pad 130, the wicking pad 140, and the connection pad 150. The support 160 is a material capable of supporting the loading pad 120, the sample pad 110, the amplification pad 130, the wicking pad 140, and the connection pad 150, and may be formed of any material having liquid impermeability (liquid nonabsorption) so that a diagnostic target molecule to be diffused and a washing buffer are not leaked.

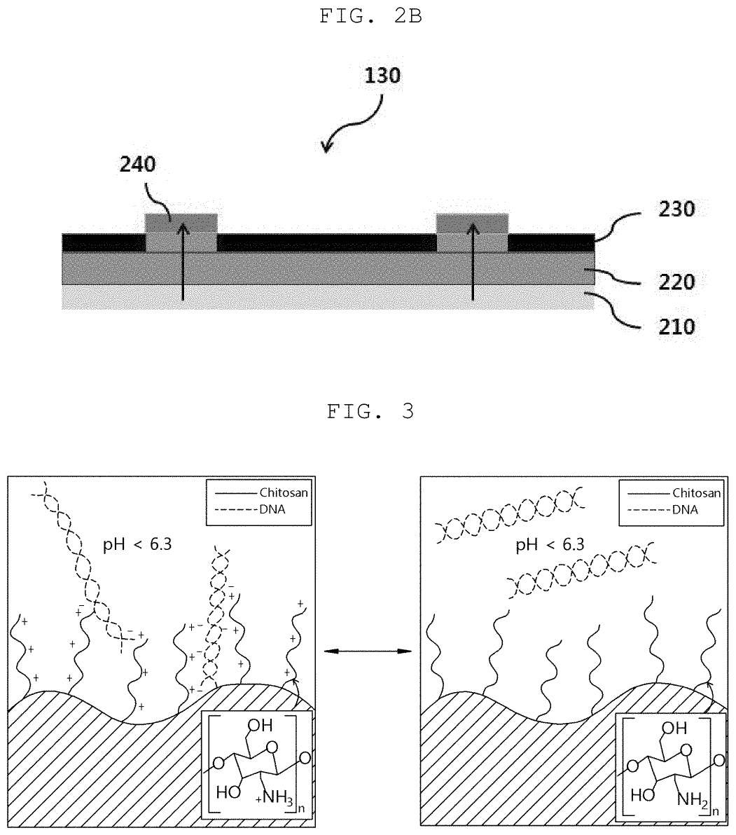

FIGS. 2A and 2B are diagrams illustrating the amplification pad 130 of the molecular diagnostics device 100 according to an embodiment of the present disclosure.

Referring to FIGS. 2A and 2B, the amplification pad 130 may include a binding pad 210, a transfer pad 220, a channel pad 230, and at least one reaction pad 240.

The binding pad 210 may include a porous membrane, and the porous membrane may specifically bind to a diagnostic target molecule. The porous membrane may be selected from the group consisting of glass fiber, silica membrane, cellulose, nitrocellulose, cellulose acetate, cotton, and nylon, but is not limited thereto. The porous membrane may be modified to specifically bind to the diagnostic target molecule, which may be appropriately selected by those skilled in the art according to a diagnostic target molecule to be detected, and for example, may be a carbohydrate-based material or a functional polymer material with a functional group.

In a specific embodiment, the surface of the porous membrane of the binding pad may be modified to form anions. When the diagnostic target molecule is a nucleic acid, a “salt bridge” is formed between an anion on the surface of the porous membrane and an anion of the nucleic acid together with a high concentration of salt, so that the binding pad may capture the diagnostic target molecule.

The salt may be a chaotropic salt, which serves to form a bond between the nucleic acid and the porous membrane, and may also affect the inactivation of cell components, particularly inactivation of proteins and dissolution of the cell membrane. As the chaotropic salt, any salt capable of making the action may be used, and guanidine thiocyanate, sodium thiocyanate, guanidine hydrochloride (guanidine HCl), sodium iodide, sodium perchlorate, and the like may be exemplified. The chaotropic may be used as a single salt, or a mixture of two or more salts may be used.

In addition, the binding pad 210 may further include a binding material that specifically binds to the diagnostic target molecule, and may extract or capture the diagnostic target molecule from a sample transferred by the washing buffer using the binding material. In an embodiment, the binding pad 210 may be made of a porous material, and the binding material may be physically coupled between pores of the binding pad 210. For example, the binding material may be added to the binding pad 210 in a state of being dissolved in a specific buffer and then dried at room temperature to bind to the inside of the binding pad 210.

The binding material may be selected from the group consisting of nucleic acid, aptamer, hapten, locked nucleic acid (LNA), antibody, antigen, DNA or RNA binding protein, single strand binding protein, Rec A protein, polypeptide, Fab fragment small-molecular chemical material, cationic compound, synthetic polymer, and mixtures thereof, and may use any material which specifically binds to the diagnostic target molecule to be detected.

The nucleic acid may serve as a binding material by binding to DNA or RNA binding proteins, such as ribosomes, polymerase, histone, gyrase, exonuclease, etc.

The antibody includes not only full-length antibody molecules, but also fragments of antibody molecules that retain antigen-binding ability, for example, antigen-binding active fragments such as active fragments [F(ab′)2, Fab, Fv, and Fd].

The terms of polypeptide, peptide, and protein may be used interchangeably to refer to a polymer of amino acid residues.

The small-molecular chemical material may be a natural compound or a synthetic compound, and may use any compound that specifically binds to a diagnostic target molecule to be detected, and for example, an intercalator, an acrylic compound, chloroquinine, quinine, novanathrone, or doxorubicin, but is not limited thereto.

The cationic compound includes all types of compounds capable of forming a complex by specifically binding to the diagnostic target molecule by electrostatic interaction with the diagnostic target molecule, and for example, may be a cationic lipid and a polymer type.

The cationic lipid may be one or a combination of two or more selected from the group consisting of N,N-dioleyl-N,N-dimethylammonium chloride (DODAC), N,N-distearyl-N,N-dimethylammonium bromide (DDAB), N-(1-(2,3-dioleoyloxy)propyl-N,N,N-trimethylammonium chloride (DOTAP), N,N-dimethyl-(2,3-dioleoyloxy)propylamine (DODMA), N,N,N-trimethyl-(2,3-dioleoyloxy)propylamine (DOTMA), 1,2-diacyl-3-trimethylammonium-propane (TAP), 1,2-diacyl-3-dimethylammonium-propane (DAP), 3beta-[N-(N′,N′,N′-trimethylaminoethane)carbamoyl]cholesterol (TC-cholesterol), 3beta-[N-(N′,N′-dimethylaminoethane)carbamoyl]cholesterol (DC-cholesterol), 3beta-[N-(N′-monomethylaminoethane)carbamoyl]cholesterol (MC-cholesterol), 3beta-[N-(aminoethane)carbamoyl]cholesterol (AC-cholesterol), cholesteryloxypropan-1-amine (COPA), N-(N′-aminoethane)carbamoylpropanoic tocopherol (AC-tocopherol) and N-(N′-methylaminoethane)carbamoylpropanoic tocopherol (MC-tocopherol).

The cationic polymer may be at least one selected from the group consisting of chitosan, glycol chitosan, protamine, polylysine, polyarginine, polyamidoamine (PAMAM), polyethylenimine, dextran, hyaluronic acid, albumin, polymer polyethyleneimine (PEI), polyamine, and polyvinyl amine (PVAm), preferably chitosan.

The transfer pad 220 is characterized by including an elution buffer in a dry state. The elution buffer refers to a buffer used to elute a diagnostic target molecule from a binding material. The elution buffer separates the diagnostic target molecule from the binding material by changing the ionic strength or pH of the buffer using an appropriate binding condition of a complex of the binding material and the diagnostic target molecule, adding a competitive molecule to a ligand (diagnostic target molecule), changing hydrophobicity of the molecule, or changing chemical properties (e.g., charge) of the ligand, so that the diagnostic target molecule cannot bind to the binding material.

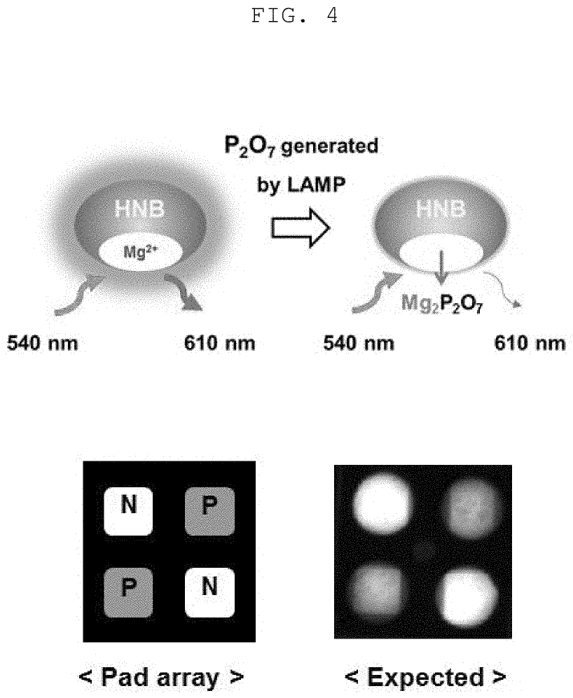

Referring to FIG. 3 for a specific embodiment, chitosan, one of the cationic polymers binds to the binding pad 210, which is a porous material, to have a positive charge (+) at a pH lower than a critical pH. When the pH of chitosan is lower than the critical pH, a positive charge of chitosan binds to a negative charge (−) of the diagnostic target molecule, so that chitosan and the diagnostic target molecule may bind to each other.

On the other hand, when the pH of chitosan is greater than the critical pH by the elution buffer added to the transfer pad 220, the positive charge of chitosan is removed and the chitosan and the diagnostic target molecule are separated so that the diagnostic target molecule may move freely. That is, the elution buffer included in the transfer pad 220 may be used to adjust the pH of the chitosan included in the binding pad 210. In an embodiment, the elution buffer may be included in the transfer pad 220 in a dry state.

The transfer pad 220 is disposed on the upper surface of the binding pad 210, and includes an elution buffer for separating the diagnostic target molecule from the binding pad in a dry state, and may transfer the diagnostic target molecule from the binding pad 210. Thereafter, the separated diagnostic target molecule may sequentially move from the binding pad 210 to the transfer pad 220, the channel pad 230, and the reaction pad 240.

In an embodiment, the transfer pad 220 may be formed of a membrane having an asymmetric structure in which a pore size decreases in a lower direction. That is, the transfer pad 220 may be constituted by an asymmetric membrane in which the pore size is reduced in the lower direction so that the diagnostic target molecule may be spread well as a whole, and accordingly, the diagnostic target molecule is spread toward a small pore to be spread evenly to the transfer pad 220.

The channel pad 230 may pass the diagnostic target molecule transferred from the transfer pad 220 through a molecule passage channel to transfer the diagnostic target molecule to the reaction pad 240. The channel pad 230 is disposed on the upper surface of the transfer pad 220 and may include at least one molecule passage channel which passes through the diagnostic target molecule received from the transfer pad 220. That is, in the channel pad 230, a molecule passage channel through which the diagnostic target molecule may pass may be formed at a position corresponding to each of the at least one reaction pad 240.

The reaction pad 240 is disposed at a position corresponding to at least one molecule passage channel, and an amplification reaction for the diagnostic target molecule may occur. In an embodiment, the reaction pad 240 may include a reaction reagent for amplifying and detecting the diagnostic target molecule in a dry state.

When the diagnostic target molecule is a nucleic acid, the reaction reagent includes a primer that specifically binds to the diagnostic target molecule, a dNTP, a reaction buffer, a recombinant enzyme, and an indicator of which fluorescence changes according to the amplification reaction.

The primer for the amplification reaction, the dNTP, the reaction buffer, recombinant enzyme, and the indicator of which fluorescence changes according to the amplification reaction may be included in the reaction pad in a dry state, and the dNTP, the recombinant enzyme, and the indicator may also be included in the transfer pad in a dry state.

In addition, the reaction pad may further include (NH₄)₂SO₄ or KCl in a dry state for forming and maintaining the structure of the diagnostic target molecule.

In addition, the reaction pad may further include a dry enhancer for accelerating the amplification reaction, for example, a surfactant.

The enhancer contributes to the success of the reaction to produce GC-rich products. In general, various enhancers may be generally included in PCR reactions to increase yield, specificity and consistency, and these enhancers may act by lowering the Tm of the template DNA. The enhancers may function through other mechanisms, including helix destabilization, neutralization of reaction inhibitors, or unknown mechanisms. The enhancers may include betaine, betaine analogs, glycerol, bovine serum albumin (BSA), polyethylene glycol, tetramethylammonium chloride, 7-deaza-GTP, neutral surfactant, dimethyl sulfoxide (DMSO), methanol, ethanol, isopropanol, formamide, acetone, acetamide, N-methylformamide, N,N-dimethylformamide, acetone, acetimide, N-methylacetimide, N,N-dimethylacetimide, 2-pyrrolidone, N-methylpyrrolidone, propionamide, and isobutyramide, but are not limited thereto. The neutral surfactants may include TWEEN-20, β-octyl-glucoside, octyl-β-thio-glucopyranoside, Triton X-100, Triton X-114, NP-40, Brij-35, Brij-58, Tween-80, Pluronic F-68, Pluronic F-127, Deoxy Big CHAP, CHAPS, CHES, nonylphenoxylpolyethoxylethanol (Tergitol type NP-40) and octylphenoxylpolyethoxylethanol (Igepal CA-630), but are not limited thereto. The betaine analogs may include homodeanol betaine, deanol betaine, propio betaine, homoglycerol Betaine, diethanol Homobetaine, triethanol homobetaine, hydroxypropyl homobetaine, N-methyl-N-(2-carboxyethyl)morpholinium intramolecular salt, N-methyl-N-(2-carboxyethyl)piperidinium intramolecular salt, N-methyl-N-(2-carboxyethyl)pyrrolidinium intramolecular salt, N,N-dimethyl-N-(2-hydroxyethyl)-N-(2-sulfoethyl)ammonium intramolecular salt, N,N-dimethyl-N-(2-hydroxyethyl)-N-(3-sulfopropyl)ammonium intramolecular salt, N,N-dihydroxyethyl-N-methyl-N-(3-sulfopropyl)ammonium intramolecular salt, N,N-dimethyl-N-(2-hydroxyethyl)-N-(4-sulfobutyl)ammonium intramolecular salt, N-methyl-N-(3-sulfopropyl)morpholinium intramolecular salt and N-methyl-N-(3-sulfopropyl)piperidinium intramolecular salt, but are not limited thereto.

In addition, the reaction pad may further include dry MgSO₄ for controlling the fluorescence intensity as the amplification reaction occurs.

The primer refers to an oligonucleotide that is a short sequence of nucleotide, and an oligonucleotide that is specifically attached to a complementary position of an opposite strand of a target DNA aptamer to initiate gene amplification.

The amplification step may use known DNA amplification methods including PCR and real-time PCR amplification methods, but in order to detect a selective nucleic acid with high sensitivity in a short time for molecular diagnostics, it is preferable to use an isothermal amplification reaction.

The isothermal amplification reaction may be performed by any one method selected from the group consisting of helicase-dependent amplification (HAD), recombinase polymerase amplification (RPA), rolling circle amplification (RCA), loop mediated isothermal amplification (LAMP), nucleic acid sequence-based amplification (NASBA), transcription mediated amplification (TMA), signal mediated amplification of RNA technology (SMART), strand displacement amplification (SDA), isothermal multiple displacement amplification (IMDA), single primer isothermal amplification (SPIA) and circular helicase dependent amplification (cHDA), preferably recombinase polymerase amplification (RPA).

The recombinant enzyme may be derived from prokaryotic, viral or eukaryotic origin. Examples of the recombinant enzyme include RecA and UvsX (e.g., RecA protein or UvsX protein obtained from any species), and fragments or mutants thereof, and combinations thereof. The RecA and UvsX proteins may be obtained from any species. In addition, the RecA and UvsX fragments or mutant proteins may also be produced using available RecA and UvsS proteins and nucleic acid sequences, and molecular biology techniques.

The indicator is a label for detection of an amplification product, and fluorescence may increase or decrease by the amplification reaction. For example, the intercalator does not bind to single-stranded DNA (ssDNA), but emits fluorescence when binding to double-stranded DNA (dsDNA) formed by synthesizing DNA (extension) after primer binding (annealing). Therefore, the intercalator fluorescent material may quantify an amplification product only by measuring the fluorescence in the annealing or DNA synthesis step.

The intercalator may be selected from the group consisting of EvaGreen, exa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Cy2, Cy3.18, Cy3.5, Cy3, Cy5.18, Cy5.5, Cy5, Cy7, Oregon Green, Oregon Green 488-X, Oregon Green 488, Oregon Green 500, Oregon Green 514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green, YO-PRO-1, YO-PRO-3, YOYO-1, YOYO-3 thiazole orange or ethidium bromide, but is not limited thereto.

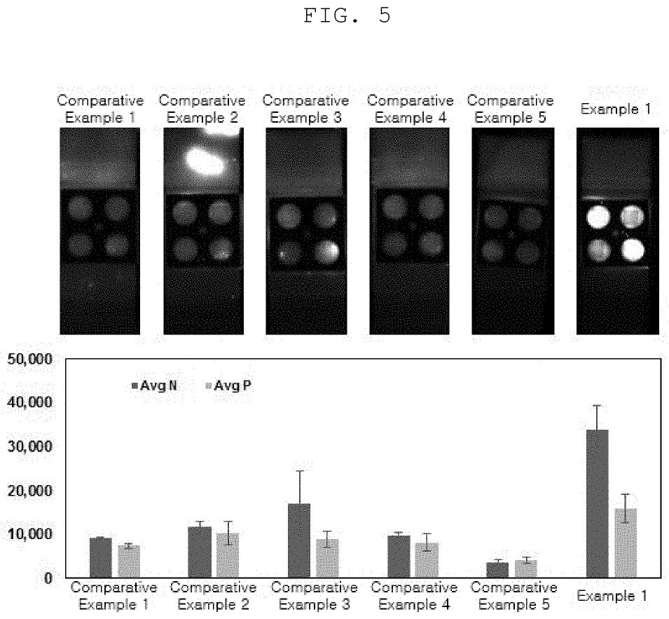

In an embodiment, referring to FIG. 4, the fluorescence of the indicator may be reduced according to an amplification reaction of the diagnostic target molecule. Accordingly, when the diagnostic target molecule is transferred to the reaction pad 240, the amplification reaction occurs and the fluorescence of the indicator decreases, thereby confirming the presence of the diagnostic target molecule. For example, the indicator of changing the fluorescence may include hydroxynaphthol blue (HNB). Hydroxynaphthol blue (HNB) is a dye that changes color in response to a concentration of magnesium ions in a reaction solution, and for example, due to the amplification of nucleic acids, Mg²⁺ binds to pyrophosphate, an amplification by-product of nucleic acid, to generate magnesium pyrophosphate. As the concentration of Mg²⁺ in the buffer is decreased, the color of the dye changes from purple to blue, particularly can be detected with the naked eye, so that the convenience is increased.

In an embodiment, a primer included in the reaction pad 240, which is a negative (N) pad, is not matched with the diagnostic target molecule, so that the amplification reaction does not occur and the fluorescence reduction of the indicator may not be shown. On the other hand, a primer included in the reaction pad 240, which is a positive (P) pad, is matched with the diagnostic target molecule, and as the amplification reaction occurs, the fluorescence reduction of the indicator may be shown.

For example, the amplification reaction may include an isothermal amplification (LAMP) reaction, and when the LAMP reaction occurs, the concentration of Mg²⁺ ions is decreased to reduce the fluorescence intensity of the indicator, and when the LAMP reaction does not occur, the concentration of Mg²⁺ ions is maintained to maintain the fluorescence intensity of the indicator.

In an embodiment, at least one of materials shown in Table 1 below is added to the amplification pad 130 according to the present disclosure in a state dissolved in a specific buffer, and then dried at room temperature and included in the amplification pad 130 in a dry state.

TABLE 1

| | | | | | Whether |
|---|---|---|---|---|---|
| | | | | | necessarily |
| | | Dry | Concentration | | included |
| Material | Function | position | range | | or not |
| Indicator | Fluorescence | Transfer | 200 to 300 | μM | ◯ |
| | intensity | pad | | | |
| | control | Reaction | | | |
| | | pad | | | |
| Primer | Isothermal | Reaction | 1-10 | μM | ◯ |
| | amplification | pad | | | |
| | reaction | | | | |
| Amplification | Reverse | Transfer | 0.1-1 | U | ◯ |
| reaction | transcription | pad | | | |
| enzyme | DNA | Reaction | | | |
| | amplification | pad | | | |
| Buffer | pH adjustment | Transfer | 10-40 | mM | ◯ |
| | | pad | | | |
| KCl | Formation of | Transfer | 10-50 | mM | X |
| | structure of | pad | | | |
| | diagnostic | | | | |
| | target | | | | |
| | molecule | | | | |
| (NH₄)₂SO₄ | Formation of | Reaction | 10-20 | mM | ◯ |
| | structure of | pad | | | |
| | diagnostic | | | | |
| | target | | | | |
| | molecule | | | | |
| Surfactant | Acceleration | Injection | 0.1-2.0% | | X |
| | of on-ionic | | | | |
| | surfactant | | | | |
| | LAMP reaction | | | | |
| Betaine | Acceleration | Transfer | 0.8-1.2M | | ◯ |
| | of | pad | | | |
| | amplification | | | | |
| MgSO₄ | Control of | Transfer | 6-8 | mM | ◯ |
| | cofactor | pad | | | |
| | fluorescence of | Reaction | | | |
| | amplification | pad | | | |
| | reaction | | | | |
| | enzyme | | | | |
| dNTPs | Synthetic | Transfer | 0.5-1.4 | mM | ◯ |
| | block | pad | | | |
| | formation of | Reaction | | | |
| | diagnostic | pad | | | |
| | target | | | | |
| | molecule | | | | |

In <Table 1>, the amplification reaction enzyme may include a polymerase. In an embodiment, KCl and (NH₄)₂SO₄ may be used to form a secondary structure of DNA, which is a molecule to be analyzed. In an embodiment, dNTPs may be used to form a DNA synthesis block, which is a molecule to be analyzed. In an embodiment, the surfactant may be injected into the sample pad 110 along with a sample including the diagnostic target molecule.

In an embodiment, the indicator, the amplification reaction enzyme, MgSO₄ and dNTPs may be included in at least one of the transfer pad 220 and the reaction pad 240 in a dry state.

FIG. 5 is a diagram illustrating molecular fluorescence images and fluorescence intensity graphs according to a configuration of the transfer pad 220 according to an embodiment of the present disclosure.

Referring to FIG. 5, the transfer pad 220 may be formed of various materials and structures in order to efficiently move a diagnostic target molecule from the transfer pad 220 to the reaction pad 240.

In this case, in Comparative Example 1, the transfer pad 220 is formed of 0.5 μm of polythersulfone (PES), and may have a symmetrical structure having the same pore sizes. In Comparative Example 2, the transfer pad 220 is formed of 5 μm of PES, and may have a symmetrical structure having the same pore sizes. In Comparative Example 3, the transfer pad 220 is formed of a first plasma membrane and may have an asymmetric structure in which the pore sizes are decreased in an upper direction. In Comparative Example 4, the transfer pad 220 is formed of a first plasma membrane and may have an asymmetric structure in which the pore sizes are decreased in a lower direction. In Comparative Example 5, the transfer pad 220 is formed of a second plasma membrane and may have an asymmetric structure in which the pore sizes are decreased in an upper direction.

In Example 1 according to the present disclosure, the transfer pad 220 is formed of a second plasma membrane and may have an asymmetric structure in which the pore sizes are decreased in a lower direction. In this case, in Example 1 according to the present disclosure, it can be seen that a fluorescence image of the diagnostic target molecule is observed the brightest, and the fluorescence intensity also has the largest value.

In addition, it can be confirmed that a difference in fluorescence intensity of the reaction pad 240 as the N pad and the reaction pad 240 as the P pad is the largest, that is, a change in fluorescence intensity is the largest, and through this, it can be more clearly confirmed that the amplification reaction occurs. In an embodiment, the second plasma membrane may have a larger difference in pore size in the lower direction than the first plasma membrane.

The above description is just illustrative of the technical idea of the present disclosure, and various changes and modifications can be made within the scope without departing from the essential characteristics of the present disclosure.

Therefore, the embodiments of the present disclosure are provided for illustrative purposes only but not intended to limit the technical concept of the present disclosure. The scope of the technical concept of the present disclosure is not limited thereto.

The protective scope of the present disclosure should be construed based on the following claims, and all the techniques in the equivalent scope thereof should be construed as falling within the scope of the present disclosure.

What is claimed is:

1. A molecular diagnostics device comprising:
a binding pad specifically binding to a diagnostic target molecule, a transfer pad including an elution buffer in a dry state that separates the diagnostic target molecule from the binding pad, and an amplification pad where an amplification reaction for the diagnostic target molecule occurs.

2. The molecular diagnostics device of claim 1, wherein the binding pad includes a porous membrane.

3. The molecular diagnostics device of claim 2, wherein the porous membrane is at least one selected from the group consisting of glass fiber, silica membrane, cellulose, nitrocellulose, cellulose acetate, cotton, and nylon.

4. The molecular diagnostics device of claim 1, wherein the binding pad further includes a binding material that specifically binds to the diagnostic target molecule.

5. The molecular diagnostics device of claim 4, wherein the binding material is at least one selected from the group consisting of nucleic acid, aptamer, hapten, locked nucleic acid (LNA), antibody, antigen, DNA or RNA binding protein, single strand binding protein, Rec A protein, polypeptide, Fab fragment small-molecular chemical material, cationic compound, synthetic polymer, and mixtures thereof.

6. The molecular diagnostics device of claim 5, wherein the cationic compound is a cationic lipid or cationic polymer.

7. The molecular diagnostics device of claim 1, wherein the amplification pad comprises a binding pad that specifically binds to a diagnostic target molecule transferred by a washing buffer to extract the diagnostic target molecule;
a transfer pad disposed on an upper surface of the binding pad and receiving the diagnostic target molecule separated from the binding pad;
a channel pad disposed on an upper surface of the transfer pad and including at least one molecule passage channel through which the diagnostic target molecule received from the transfer pad passes; and
at least one reaction pad disposed at a position corresponding to the at least one molecule passage channel and generating an amplification reaction for the diagnostic target molecule.

8. The molecular diagnostics device of claim 1, further comprising:
a sample pad in which a sample containing the diagnostic target molecule is accommodated;
a loading pad in which a washing buffer carrying the diagnostic target molecule is accommodated; and
a wicking pad which absorbs the washing buffer developed into the amplification pad and is positioned downstream of the amplification pad.

9. The molecular diagnostics device of claim 8, wherein the loading pad, the sample pad, the amplification pad, and the wicking pad are sequentially spaced apart from each other and arranged.

10. The molecular diagnostics device of claim 7, wherein the washing buffer includes at least one of distilled water (D.W.) and deionized water (D.I water).

11. The molecular diagnostics device of claim 7, wherein the reaction pad includes a dry primer for generating the amplification reaction for the diagnostic target molecule.

12. The molecular diagnostics device of claim 7, wherein at least one of the transfer pad and the reaction pad includes a dry indicator of which fluorescence intensity changes as the amplification reaction occurs.

13. The molecular diagnostics device of claim 7, wherein at least one of the transfer pad and the reaction pad includes a dry amplification reaction enzyme for generating the amplification reaction for the diagnostic target molecule.

14. The molecular diagnostics device of claim 7, wherein the amplification reaction is an isothermal amplification reaction.

15. The molecular diagnostics device of claim 14, wherein the isothermal amplification reaction is at least one selected from the group consisting of helicase-dependent amplification (HAD), recombinase polymerase amplification (RPA), rolling circle amplification (RCA), loop mediated isothermal amplification (LAMP), nucleic acid sequence-based amplification (NASBA), transcription mediated amplification (TMA), signal mediated amplification of RNA technology (SMART), strand displacement amplification (SDA), isothermal multiple displacement amplification (IMDA), single primer isothermal amplification (SPIA), and circular helicase dependent amplification (cHDA).

16. The molecular diagnostics device of claim 7, wherein the amplification pad or the transfer pad includes an enhancer in a dry state for accelerating the amplification reaction.

17. The molecular diagnostics device of claim 7, wherein the reaction pad further includes dry MgSO4 for controlling the fluorescence intensity as the amplification reaction occurs.

18. The molecular diagnostics device of claim 7, wherein at least one of the transfer pad and the reaction pad includes dry dNTPs for forming a synthesis block of the diagnostic target molecule.

19. The molecular diagnostics device of claim 1, wherein the transfer pad is formed of a membrane having a structure in which the pore sizes decrease in a lower direction.
